(19) United States
(12) Patent Application Publication
(10) Pub. No.: US 2022/0151829 A1
(43) Pub. Date: May 19, 2022

(54) Liquid Dropper Device

(71) Applicant: J4J, LLC, Twin Falls, ID (US)

(72) Inventor: Jared Henderson, Twin Falls, ID (US)

(21) Appl. No.: 17/526,644

(22) Filed: Nov. 15, 2021

Related U.S. Application Data
(60) Provisional application No. 63/113,696, filed on Nov. 13, 2020.

(51) Int. Cl. A61F 9/00

(52) U.S. Cl. CPC A61F 9/0026

(57) ABSTRACT

The device relate to liquid dropper that will enable an individual to precisely instill a liquid medium onto a surface in many orientations. The device will preferably hold a specified volume droplet on the end and present it to be taken up to desired application by adhesion forces. Employed in the device is a non-mechanical valve to keep contents from refluxing back into the parent medium. This may be device may be employed in many different applications but will be adapted to an eye dropper below. The function of this device is to form a droplet on the end of said dispensing device while using a non-mechanical valve to separate the desired droplet from parent medium. Hydrophobic forces along with vent valves are utilized to accomplish “droplet splitting off” preserving the parent medium. Further disclosed are alternate tips of eye drop bottles or attachments to bottles that can be utilized with or without vented delivery tubes.

### PRIORITY/CROSS-REFERENCE TO RELATED APPLICATIONS

This application claims the benefit of U.S. Provisional Application No. 63/113,696, filed Nov. 13, 2020 the disclosure of which is incorporated by reference.

### TECHNICAL FIELD

The disclosure generally relates to the field of dispensing liquid drops. Particular embodiments relate to improved eye drop applicator devices and other devices that dispense liquid from a reservoir wherein dispensing of a liquid drop will serve to maintain the integrity of remaining liquid in the reservoir.

### BACKGROUND

Maintaining sterility and/or taking measures to reduce contamination is an important consideration in dispensing of one or more drops of liquid from a reservoir, such as in pipetting or eye drop applications, into a desired target. When the drop(s) of liquid are exposed to atmosphere there is potential for contamination. Typically, liquid drops are formed as liquid is propelled, either through mechanical force or gravity, from a reservoir through a tube where a drop forms at the end of the tube. Typically liquid does not travel through the tube in a drop by drop fashion, and releasing of the propelling force causes a reflux of liquid back into the reservoir. If it has contacted any foreign particles, the refluxed liquid can cause contamination of the liquid in the reservoir.

Further, the application of a drip from the end of the tube onto an intended target, such as the eye of a user or into a tube, can face similar issues. For example, the instillation of eye drops is difficult and cumbersome for many individuals. Moreover those with limitations often forgo the care they need due to the mechanical barriers of taking eye drops. This eye dropper device is built to more accurately instill eye drops in the precise desired location, and to eliminate eye drop waste.

Current eye droppers utilize the “bombing method” where eye drops fall from above the eye stimulating the corneal ocular reflex. Stimulating this reflex will produce squinting after eye drops are instilled into the eye. Often freshly dispensed liquid is squeezed out after a person squints from the reflex activation as a drop hits the eye. Even the act of dropping results in drops that do not hit the eye, or partially hit the eye. Overall the efficacy of eye drops are limited because of these and other problems with application. Also conventional eye droppers often dispense more than one drop unintentionally which results in waste because less than one full drop can anatomically be held in the eye. This is evidenced by the typical “tearing” that occurs after an eye drop is instilled. It is common for users to be uncertain whether any liquid was placed in the intended place.

### SUMMARY OF THE DISCLOSURE

The purpose of the Summary is to enable the public, and especially the scientists, engineers, and practitioners in the art who are not familiar with patent or legal terms or phraseology, to determine quickly from a cursory inspection, the nature and essence of the technical disclosure of the application. The Summary is neither intended to define the inventive concept(s) of the application, which is measured by the claims, nor is it intended to be limiting as to the scope of the inventive concept(s) in any way.

The disclosed eye dropper and eye dropper attachment utilizes a lumen directly from the eye dropper nipple or terminus from the container which coveys it out to the terminal dispenser. In many cases it will serve to extend the native lumen. The device is designed such that surface tension breaks the liquid apart just at the end of the lumen before any of the liquid is able to reflux back in the bottle, also called a reservoir, to limit contamination of the contents. The tip has a design that manipulates the surface tension properties of a liquid medium to collect the droplet on the tip and present the droplet to ocular or conjunctival surface. The application of the droplet from the tip of the dispenser to the eye is facilitated by surface tension/capillary action without touching the eye or structures itself. The device preferably will be constructed of an electromeric substance like but not limited to synthetic or natural rubbers like silicone, latex, nitrile etc. Using these materials along with the fine structure of the droplet holding portion (thin walls) will help to avoid any inadvertent ocular damage in case of direct contact. Materials used may also have antibacterial properties. Materials nay be autoclavable (sterilized).

This novel dropper, which may find other applications, uses the surface tension of the water and manipulates hydrophobic and hydrophilic properties intrinsic to the materials to deliver the drop in the most efficient and convenient manner. This device can be adapted to other applications where a single drop is needed to be serially separated from a liquid medium and presented to another medium facilitated by surface tension. By changing the shape and size of the terminal end of the device it would allow the specified volume of fluid to be delivered. Operator would force a droplet into the tip in whatever position they chose and then it would be held at ready until applied to surface of eye when convenient. In the case of an eye drop bottle, the reservoir constitutes a bottle that is squeezed to dispense one or more eye drops. In the disclosed improved eyedropper squeezing of the bottle would convey the liquid to the tip of the delivery device where the droplet would be formed. Upon release of the squeezing of the bottle vent(s) would allow liquid in the lumen to retreat and droplet would remain at the tip.

Using the refraction properties of water droplets a shape and color combination would notify the user when an appropriate drop volume had accumulated in the applicator tip. Once the drop covers the colored surface it will be refracted throughout the drop.

The disclosed concepts can be utilized as an attachment for attaching to the end of a standard eye drop bottle, as a removable tip that attaches to an eye drop bottle, or as an integrally formed end of the eye drop bottle.

The eye dropper devices disclosed herein help to overcome the oculocorneal reflex and associated problems (such as squinting and lashes in the way) that limit eye drop applications. The eye dropper devices disclosed herein help to reduce the need for positioning of the head of the person or animal receiving the drops such as head tilting or leaning back and drops can be placed in an upright position. This functionality serves to reduce waste of eye drops, limit squinting after application and resulting force the pushes medication out of the eye, and allows the user to know that solution gets into the eye by direct observation.

The devices are further designed to keep contents from refluxing back into the bottle thereby limiting contamination of contents through the use of one or more vent holes forming a non mechanical valve.

Preferably hydrophobic/hydrophilic principles used to facilitate droplet transfer by capillary action/surface tension. This serves to wick droplets away from surface of the applicator end of the devices and/or to facilitate flow of droplets through the device

The principles disclosed herein can be applied to any application where droplet transfer is needed, including but not limited to pipetting.

A further benefit of the disclosed technology is that it both dispenses liquid eye drop material in drop form, as well as suspends the drop for application. The applicant is not aware of technology that is capable of both dispensing a drop and suspending the drop for application.

In one embodiment an attachment for an eye drop bottle is provided. The attachment having a first attachment end and a second attachment end separated by an elongate tube section. The elongate tube section defines a lumen configured for the flow of liquid from the eye drop bottle through the first attachment end through the lumen to the second end of the attachment. Thus when a user squeezes the eye drop bottle to release an eye drop, the drop travels through the tip of the bottle, through the attachment and forms a drop on the second end of the attachment. The second end of the attachment is preferably formed in a dish or bowl shape, including such as a shape resembling an ice-cream scooper, yet it may not be limited by any geometry that can be configured to temporary hold a liquid droplet.

At least one vent is positioned in the attachment between the first attachment end and the second attachment end. The vent is configured to vent the lumen to atmosphere. The vent is configured such that when a user releases depression on the eye drop bottle the vent allows ambient air to enter the lumen and preventing any liquid that has traveled beyond the vent to return via the lumen to the eye drop bottle. The vent can be an elongate slot, a cylinder, or a variety of shapes. Multiple vents can be utilized. Vents can be manipulated in any way such as to facilitate retention of liquid in the lumen.

The general dish like shaped second attachment end is configured such that the liquid that the liquid forms a bead on the dish. The dish is configured such that a user can hold an eye drop bottle and attached attachment in a generally vertical position with a bead of liquid from the eye drop bottle positioned on the dish and the user is able to lower the user's face and eye toward the bead. This allows the eye to contact the bead causing the liquid to travel onto the user's eye due to the surface tension of the liquid.

The lumen can be configured to facilitate separation of a drop from the remaining liquid in the attachment. For example, the lumen can utilized one or more, such as a plurality of elongate channels of varying cross section extending lengthwise in the lumen and configured to separate a drop. The lumen can use varying hydrophobic and/or hydrophilic materials and/or coatings to facilitate delivery of the drop.

The attachment can be configured to be placed over the tip of an eyedrop bottle. Alternately the attachment can be configured to connect directly to the bottle. Alternately the tip of an eyedrop bottle can be manufactured directly with the principles discussed above for the attachment.

Still other features and advantages of the presently disclosed and claimed inventive concept(s) will become readily apparent to those skilled in this art from the following detailed description describing preferred embodiments of the inventive concept(s), simply by way of illustration of the best mode contemplated by carrying out the inventive concept(s). As will be realized, the inventive concept(s) is capable of modification in various obvious respects all without departing from the inventive concept(s). Accordingly, the drawings and description of the preferred embodiments are to be regarded as illustrative in nature, and not as restrictive in nature.

### BRIEF DESCRIPTION OF THE FIGURES

FIG. 1 illustrates a partial section view of an embodiment of an eye dropper attachment attached to an eye dropper bottle.

FIG. 2 illustrates an embodiment of a lumen of an eye dropper attachment.

FIG. 3 illustrates an embodiment of an eye dropper attachment to a bottle.

FIG. 4 illustrates an embodiment of a cup for the end of an eye dropper attachment.

FIG. 5 illustrates a section view of an attachment mechanism of an eye dropper attachment to an eye dropper bottle.

FIG. 6 illustrates a section view of an eye dropper attachment.

FIG. 7 illustrates an attachment mechanism for an eye dropper attachment to an eye dropper bottle.

FIG. 8 illustrates a perspective view of an embodiment of an eye dropper attachment configured for attaching to an eye dropper bottle.

FIG. 9 illustrates an isometric view of the eye dropper attachment of FIG. 8.

FIG. 10 illustrates a section view of the eye dropper attachment of FIG. 9 along section line B.

FIG. 11 illustrates an isometric view of the eye dropper attachment of FIGS. 8-9.

FIG. 12 illustrates an isometric view of the eye dropper attachment of FIGS. 8-11 showing internal components.

FIG. 13 illustrates an alternate embodiment of an eye dropper attachment.

FIG. 14 illustrates a section view along section line A of FIG. 13.

FIG. 15 illustrates an isometric view of the eye dropper attachment of FIGS. 13-14.

FIG. 16 illustrates an alternate embodiment of an eye dropper attachment having a basket for retaining an eye drop that has formed in the cup of the attachment.

FIG. 17 illustrates a perspective view of an alternate embodiment of an eye drop attachment having a scoop or cup formed at the end in which the eye drop forms.

FIG. 18 illustrates a top view of the eye dropper attachment of FIG. 17.

FIG. 19 illustrates an isometric view showing internal components of the eye dropper attachment.

FIG. 21 illustrates a side isometric view of the attachment of FIG. 17.

FIG. 22 illustrates a section view along section line C of FIG. 21.

### DETAILED DESCRIPTION OF THE FIGURES

While the presently disclosed inventive concept(s) is susceptible of various modifications and alternative constructions, certain illustrated embodiments thereof have been shown in the drawings and will be described below in detail. It should be understood, however, that there is no intention to limit the inventive concept(s) to the specific form disclosed, but, on the contrary, the presently disclosed and claimed inventive concept(s) is to cover all modifications, alternative constructions, and equivalents falling within the spirit and scope of the inventive concept(s) as defined herein.

In the following description and in the figures, like elements are identified with like reference numerals. The use of “e.g.,” “etc,” and “or” indicates non-exclusive alternatives without limitation unless otherwise noted. The use of “including” means “including, but not limited to,” unless otherwise noted.

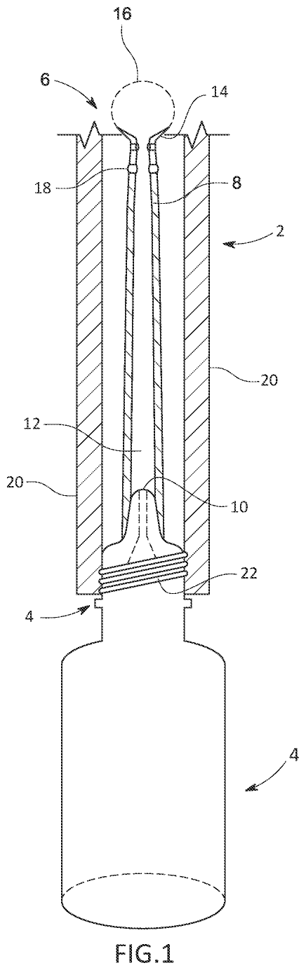

FIG. 1 illustrates an eye drop bottle attachment 2 attached to an eye drop bottle 4. Eye drop bottle attachment has a first attachment end 4 and a second attachment end. The eye drop bottle attachment has a tube 8 that extends between the first end and second end of the eye drop attachment. The first end of the eye drop attachment is configured to attach to an eye drop bottle such that fluid disbursed from the eye drop bottle from the orifice 10 is configured to travel down the lumen 12 of the eye drop bottle attachment. The second end of the eye drop bottle attachment is configured in a generally cup like shape 14 such that any liquid that is disbursed from the eye drop bottle travels down the eye drop bottle attachment lumen and forms a bead 16 at the second end of the eye drop bottle attachment. While a cup like shape is thought to be preferred, an alternate shape can be utilized such as a ball shape or other shape that will allow the liquid to bead on the end of the cup. The tube has one or more vents 18 that are configured to vent the lumen to atmosphere. The vents are configured such that when the user depresses the eye drop bottle and a bead forms at the second end of the eye drop bottle attachment, when the user releases squeezing the eye drop bottle, the vents allow air from the atmosphere the return into the lumen thus preventing the bead of liquid from returning down the lumen to the bottle. This will help prevent contamination of the liquid material in the eye drop bottle. In a preferred embodiment the eye drop bottle attachment has an outer wall 20 that encircles the eye drop bottle tube 8. In a preferred embodiment the outer wall is configured to threadingly engage with the threads 22 of an eye drop bottle. Alternatively the attachment can be constructed without the outer wall and with only the tube 8 with the first end of the tube configured to attach to the eye drop bottle. The length of the tube can vary depending on application, but a preferred length range is thought to be from 1.5 cm to 6.5 cm from attachment to the dispenser nipple to the end of the applicator.

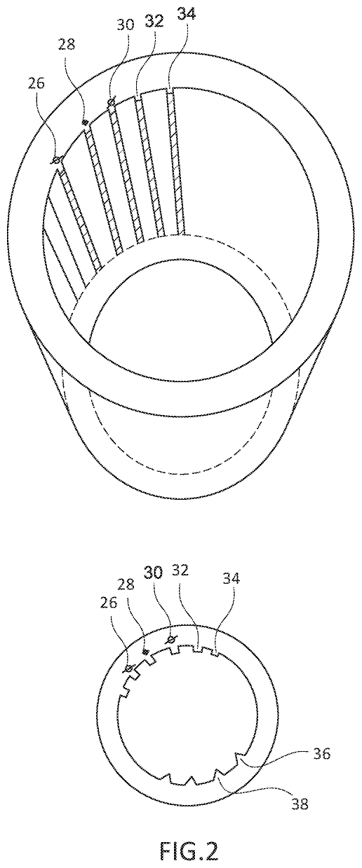

FIG. 2 illustrates a cross sectional perspective view of an embodiment of the lumen. The depicted lumen has a series of length wise channels formed by projections 26, 28, 30, 36, 34, 36, 38 have varying shape and size. These channels are shown with differing cross sections to provide differing effects on the travel of liquid through the lumen. Preferably these tubes can be made of different hydrophobicity or hydrophilicity to facility the breakage of separate drops from the eye dropper and keeping these drops separate. Alternatively the lumen wall can be smooth, or have uniform channeling and projection shape and size, depending on the intended characteristics of the lumen. Preferably the lumen is tapered to facilitate transfer of liquid from the body of the eye drop bottle to the tip of the applicator. The device, including the lumen and cup, are designed structurally so that a drop formed and applied repeatedly will not compromise the integrity of the liquid in the bottle. This provides in essence a one-wav conveyer of liquid that does not allow mixing of liquid in the bottle or reservoir with liquid that has been exposed to foreign contaminants in the process. The device produces a liquid droplet in the cup in a manner that cannot contaminate the liquid in the bottle.

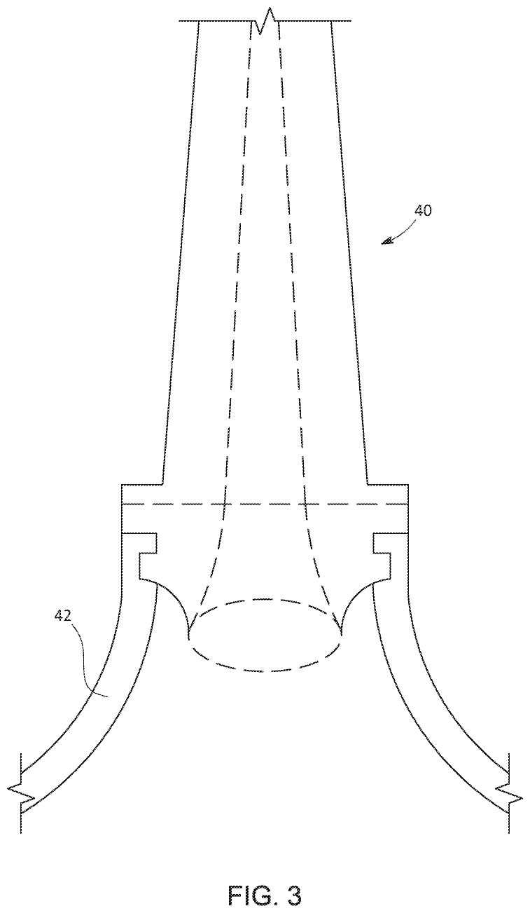

FIG. 3 illustrates an attachment tip in accordance with the inventive concepts disclosed herein. This attachment 40 snaps into an end of the eye drop bottle 42. This forms a removable piece on top of the bottle dropper. This construction allows for a disposable unit, for example for a physician to utilize with a patient and discard.

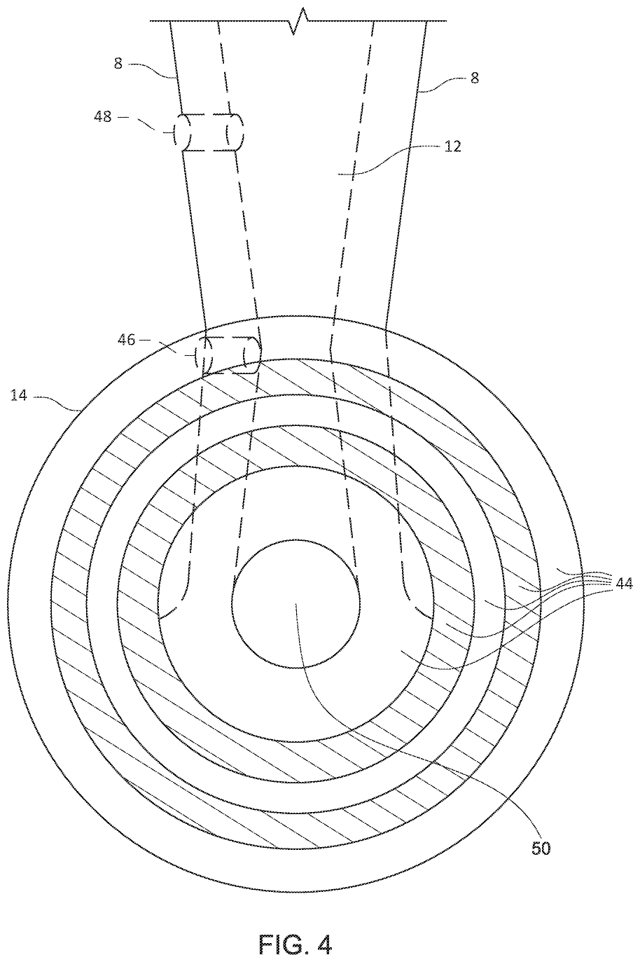

FIG. 4 illustrates a diagram of a second end of an eye drop bottle attachment having a cup 14. The depicted cup 14 is shown with rings 44 of varying hydrophobicity and hydrophilicity to facilitate the beading of the liquid from the eye drop bottle on the end cup. Preferably the cup will utilize one or more fine hydrophilic rings that will hold the droplets to the cup but will minimize the amount of residue of liquid on the cup following dispensing of the eye drop. Alternatively, or additionally, hydrophobic or hydrophilic coatings may be used with an arrangement to promote formation and dispensing of the drop with minimal residue left behind on the cup. The coatings and/or design utilizing coatings and materials of varying hydrophobicity and hydrophilicity is thought to further assist in reducing potential contamination and microbial growth by minimizing any potential liquid available for microbes to grow in. The wall 8 of the tube are shown with two cylindrical vents 46, 48. The vents allow for venting of the atmosphere to the lumen 12 when squeezing pressure on the eye drop bottle is released. The liquid travels from the eye drop bottle through the lumen 12 out of the orifice 50 where it beads into the cup 14.

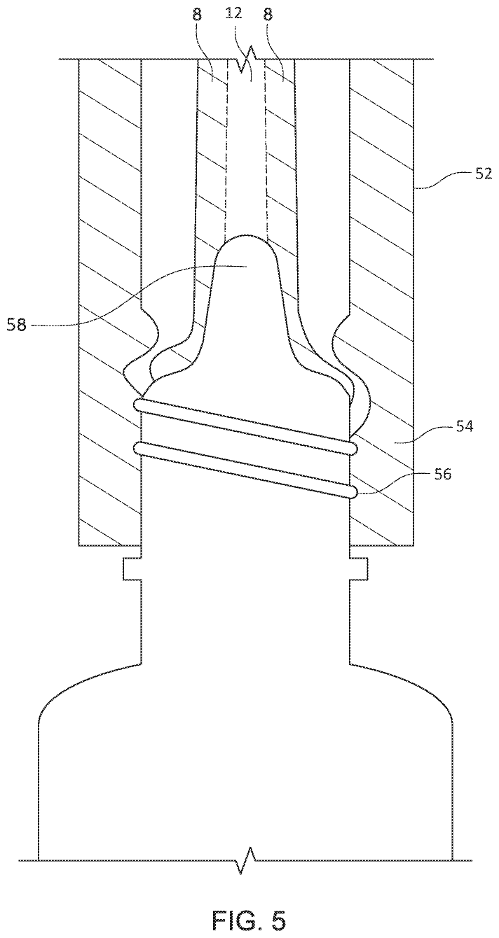

FIG. 5 illustrates an alternative mechanism for attaching the eye drop bottle attachment to an end of the eye drop bottle. The attachment utilizes an outer wall 52 for this configured to threadingly engage at a first end 54 with the threads 56 of the dispensing end of the eye drop bottle 58 the lumen 12 is in contact with the orifice in the dispensing end of the eye drop bottle. The fluid travels through the lumen defined by the wall 8 to the second end of the attachment and the cup (not shown). Preferably the tubes 8 are forms of silicon or similar materials such that a seal can be formed between the tubes and the dispensing end of the eye drop bottle.

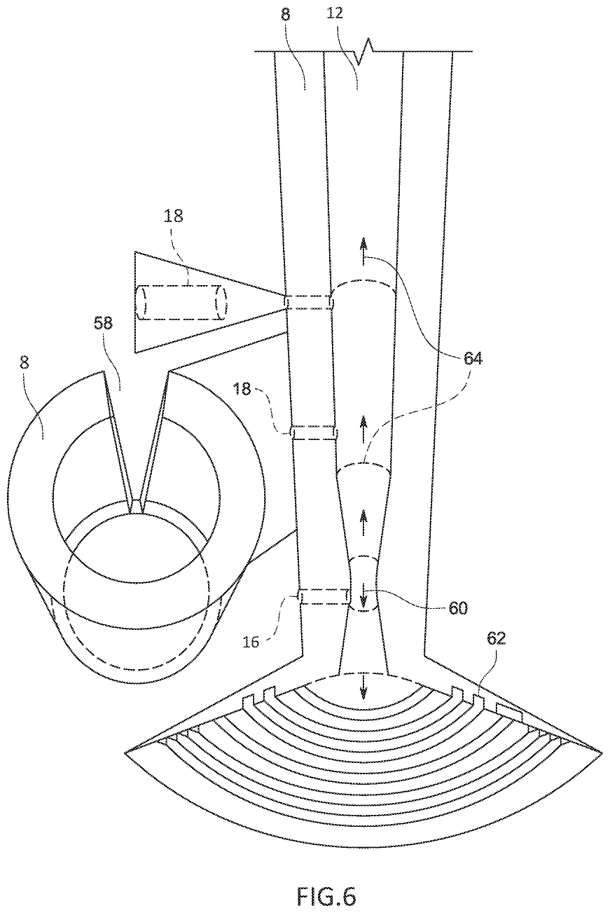

FIG. 6 illustrates a cross section of the attachment depicting the travel of fluid in the lumen 12. A series of vents are shown including an elongate vent 58 in the outer wall 8 of the attachment. When the eye drop bottle is squeezed, the fluid travels down 60 the lumen to the end of the eye drop bottle attachment where it beads up in the cup. The depicted cup has a series of raised concentric ridges 62 to facilitate the beading of the liquid in the cup. The cup can be formed with a variety of refracting material such that when liquid is formed in a bead in the cup, light refracted through the liquid and the cup causes the cup to form a different color. The wall 8 of the tube further includes cylindrical vents 18 when a user releases the squeeze on the eye drop bottle fluid that has not reached the cup is drawn back 64 down the lumen with the vents allowing air from atmosphere to disrupt the separate droplets, allowing droplets that have not reached the cup to return to the eye drop bottle.

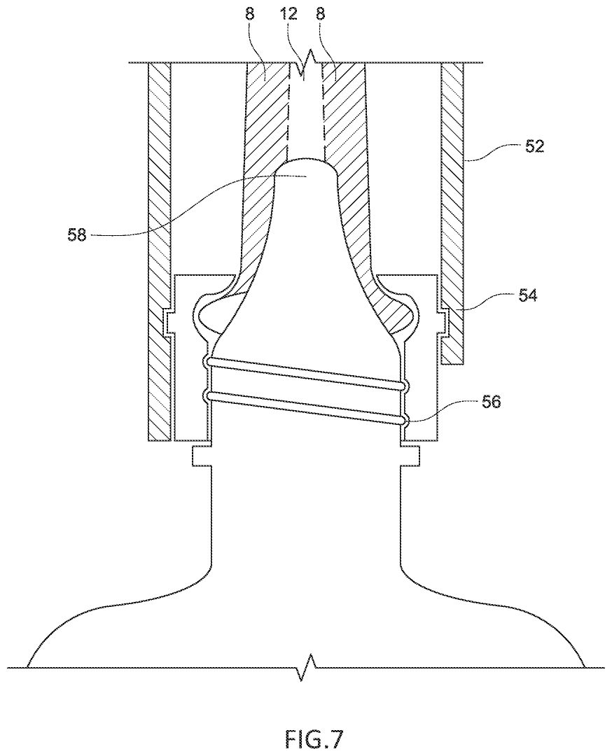

FIG. 7 illustrates an alternative attachment mechanism for the first end of the eye drop bottle attachment to connect to a dispensing end of an eye drop bottle. In the depicted embodiment the eye drop bottle attachment 2 utilizes an outer wall 20 and a tube having inner wall 8. A clamp mechanism 66 is positioned and configured to threadingly engage with the threads 68 of the dispensing end of the eye drop bottle the outer wall 20 serves to impart force inwardly on the clamp mechanism 66 such that as the attachment is threaded onto the end of the eye drop bottle, the clamp mechanism asserts a clamping mechanism on the lower ceiling end 70 of the tube wall 8 of the attachment. This serves to facilitate a liquid tight attachment between the eye drop bottle attachment and the dispensing end 58 of the eye drop bottle.

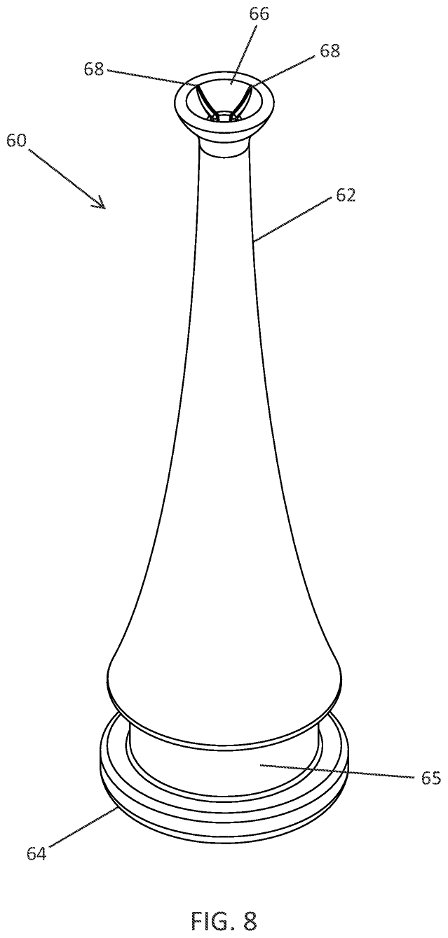

FIGS. 8-12 illustrate an embodiment of an eye dropper attachment configured to be inserted into an eye drop bottle over the original tip (alternatively called a nipple or nozzle) has been removed. FIG. 8 illustrates an embodiment of an eye dropper attachment. The eye dropper attachment 60 is formed with a neck 62 extending between a first end 64 of the eye dropper attachment. The first end of the depicted eye dropper attachment is configured with a ring 65 to retain the attachment over the tip of the eye dropper.

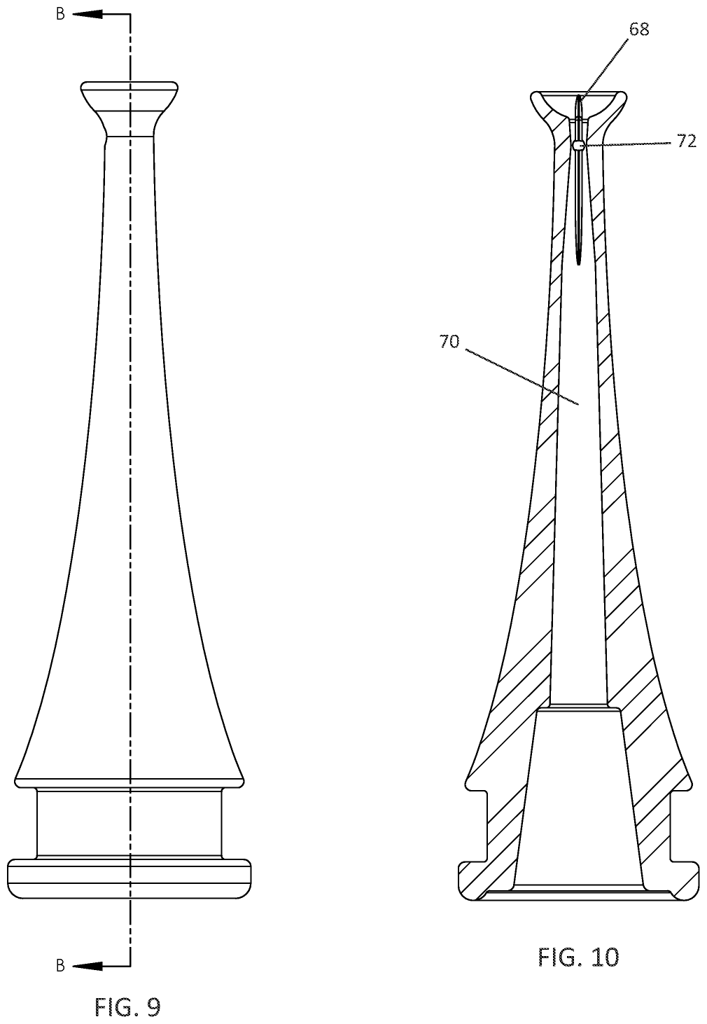

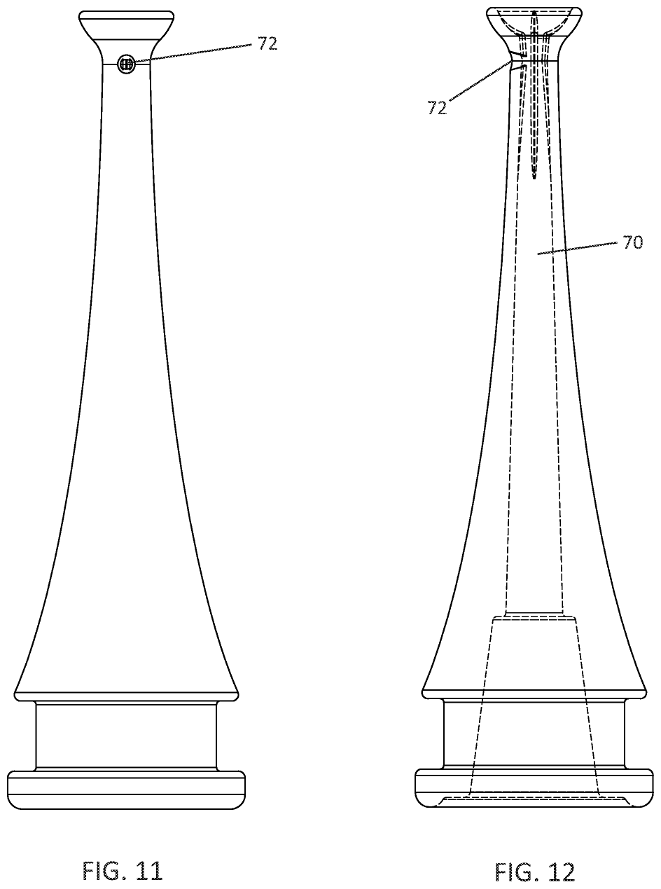

FIG. 9 illustrates an isometric view of the nozzle of FIG. 8. FIG. 10 illustrates a section view of the nozzle shown along line B of FIG. 9. The eye dropper attachment has an air vent 72 configured to allow for air from atmosphere to enter the eye dropper attachment such that when pressure on the dropper bottle is released, air is allowed into the pore 72 and breaks the drop that is in the cup 68 away from the liquid remaining in the lumen 70 of the eye dropper attachment. FIG. 11 illustrates an isometric view showing the pore on the outer surface of the attachment. FIG. 12 illustrates an isometric view showing with dotted lines the internal components.

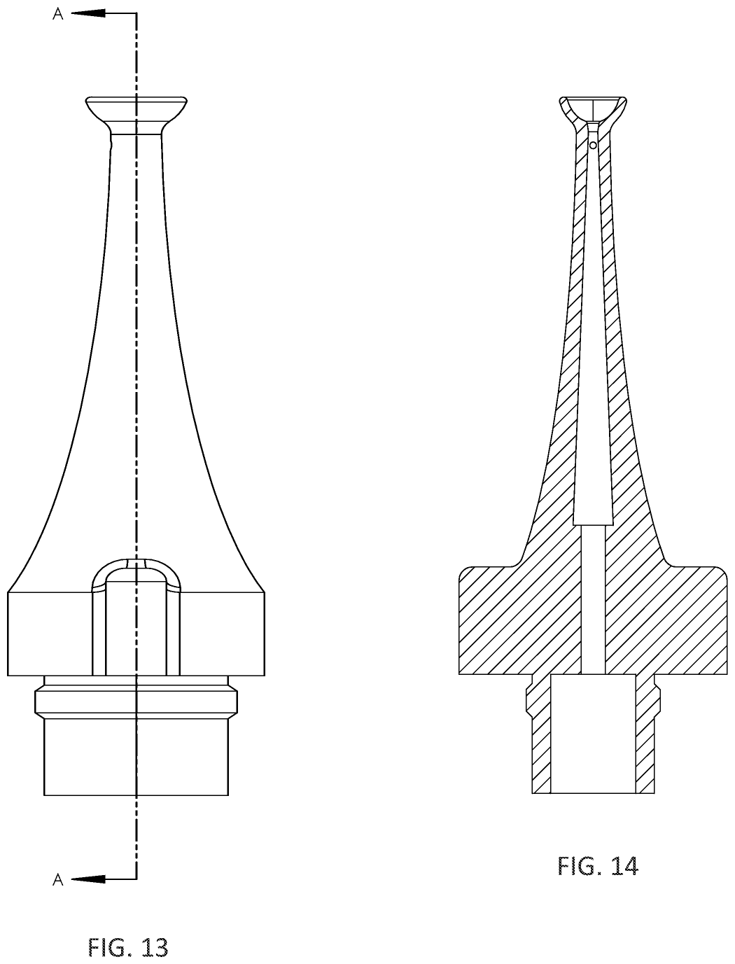

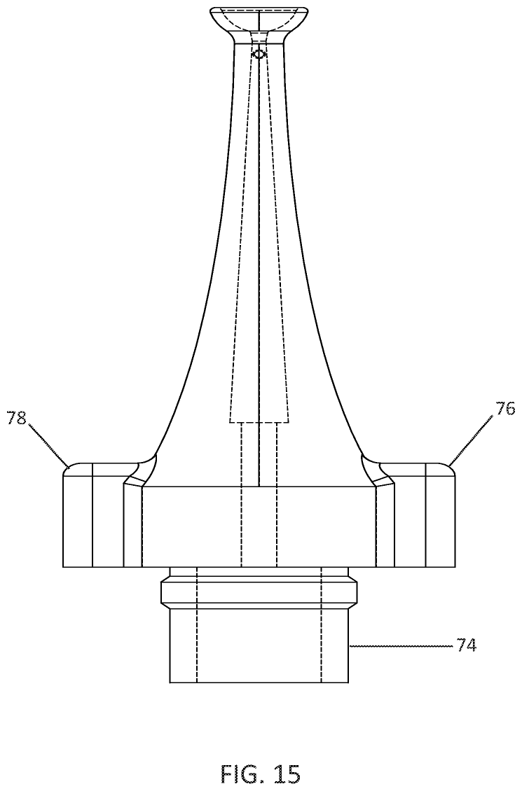

FIGS. 13-15 illustrate an embodiment of an eye dropper attachment for an eye dropper bottle that is configured for insertion into an eye dropper bottle after the original nipple or nozzle has been removed. The embodiment utilizes a stem 74 that is configured for positioning into an opening of the eye dropper bottle. Tabs 76, 78 circumferentially extend outward from the attachment. The tabs are configured to allow users thumbs and fingers to attach the device to an eye dropper bottle.

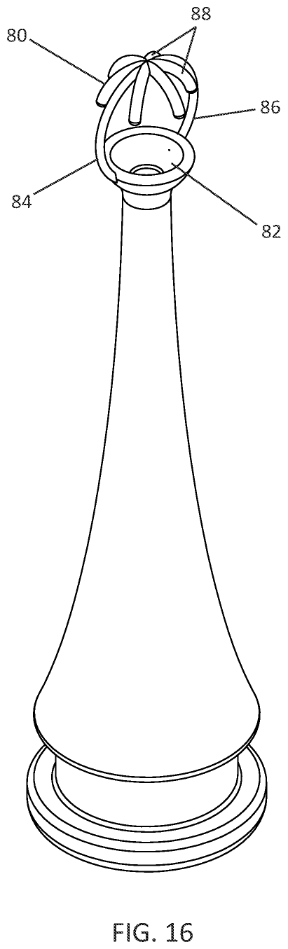

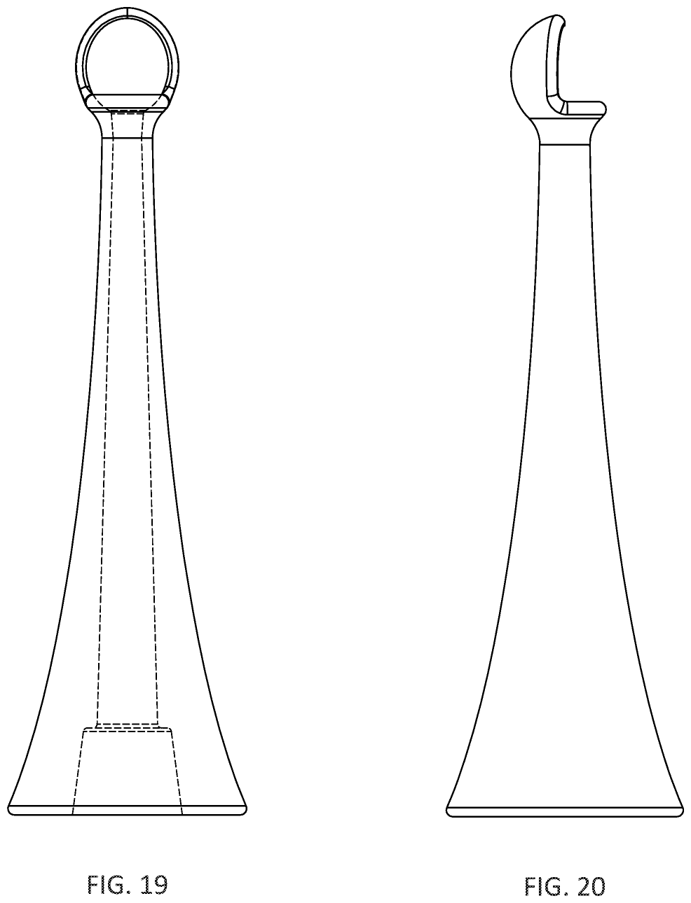

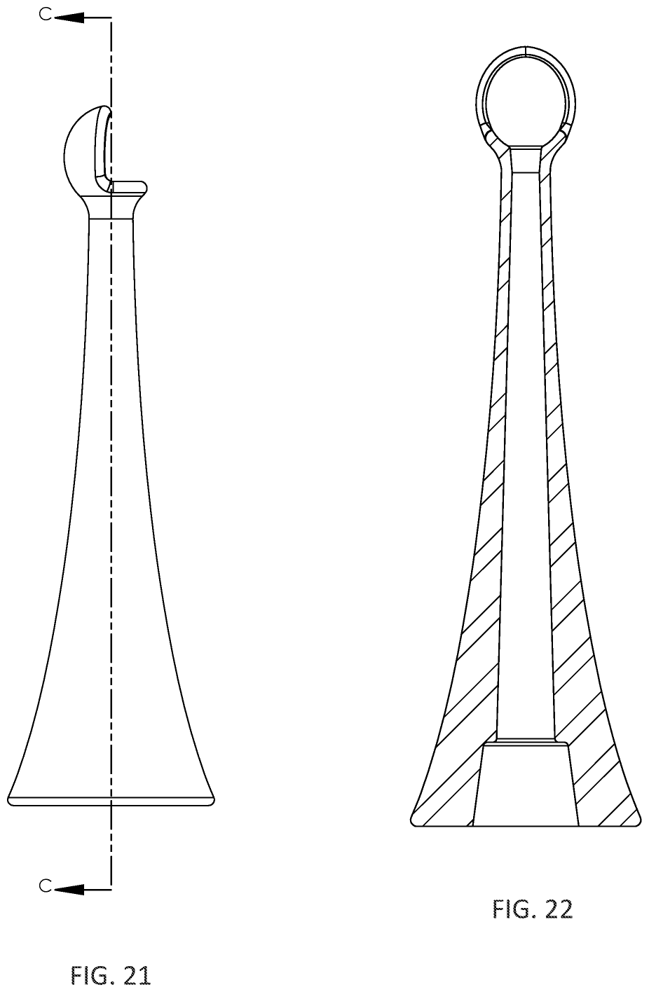

FIGS. 16-23 illustrate varying tips for eyedrop bottles and/or attachments to bottles. These tips can be used with or without vents in the lumen leading to the tips. FIG. 16 illustrates an embodiment of an eye dropper attachment having a configuration to retain an eye drop in the cup of the attachment. The end of the attachment as a cup 82. Two opposing arms 84, 86 extend away from the cup with an umbrella or inverse basket 80 positioned such that an eye drop forms in the cup and is retained in position by the finger 88 of the umbrella shape.

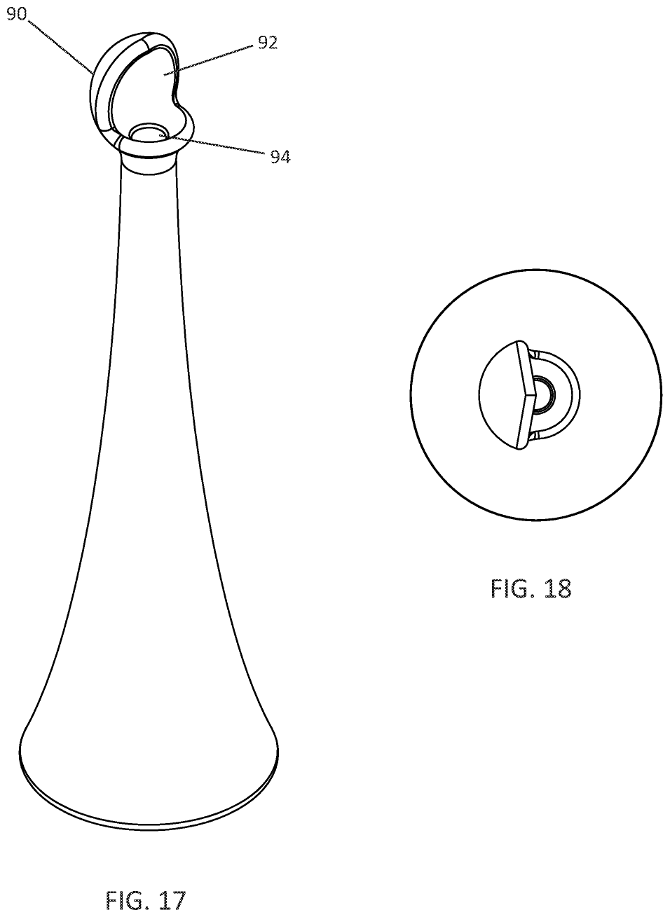

FIG. 17-22 illustrate an alternate embodiment of an end configuration for retaining an eye drop formed in the cup at the end of the eye dropper attachment. The end 90 is formed in a cup-like shape having a cup 92 formed in the appearance of a scoop. The eye drop is admitted from an opening 94 and forms in the scoop. The eye drop is maintained such that a user can raise the attachment and/or lower the users head toward the eye drop, such that the eye contacts the eye drop utilizing capillary action, the eye drop flows to the users eye.

The eye drop applicator and/or attachment can further include a removable cap intended to protect the device and to reduce contamination potential. Silicone insert would fit directly over the nipple of an eye dropper bottle. Alternatively, it could be bonded to a plastic component and have threads that would directly replace the “lid” of the original dropper and a larger cap would fit over the insert so it would not have to be replaced with each use.

Still other features and advantages of the presently disclosed and claimed inventive concept(s) will become readily apparent to those skilled in this art from the following detailed description describing preferred embodiments of the inventive concept(s), simply by way of illustration of the embodiments contemplated by carrying out the inventive concept(s). As will be realized, the inventive concept(s) is capable of modification in various obvious respects all without departing from the inventive concept(s). Accordingly, the drawings and description of the preferred embodiments are to be regarded as illustrative in nature, and not as restrictive in nature.

While certain exemplary embodiments are shown in the Figures and described in this disclosure, it is to be distinctly understood that the presently disclosed inventive concept(s) is not limited thereto but may be variously embodied to practice within the scope of this disclosure. From the foregoing description, it will be apparent that various changes may be made without departing from the spirit and scope of the disclosure as defined herein.

I claim:

1. An eye drop dispenser, said dispenser comprising:
a reservoir, said reservoir configured to retain an eye drop liquid;
a dispenser tube in fluid connection with said reservoir a first end of said dispenser tube, wherein said dispenser tube is configured to transfer a drop of said eye drop liquid from said reservoir to a second end of said dispenser tube; and
a tip, wherein said tip is positioned at said second end of said dispenser tube, wherein said tip is configured to suspend said drop such that a user is able to touch the user's eye to the suspended drop.

2. An attachment for an eye drop bottle, said attachment comprising:
a first attachment end and a second attachment end separated by an elongate tube section, wherein said elongate tube section defining a lumen configured for the flow of liquid from the eye drop bottle through said first attachment end through said lumen to said second end when a user squeezes the eye drop bottle to release an eye drop, wherein said first end is configured to attach to an eye drop bottle, wherein said second end is configured in a general dish like shape;
at least one vent positioned in a side of said elongate tube between said first attachment end and said second attachment end and configured to vent said lumen to atmosphere, wherein said vent is configured such that when a user releases depression on said eye drop bottle said vent allows ambient air to enter said lumen and preventing any liquid that has traveled beyond said vent to return via said lumen to said eye drop bottle;
wherein said general dish like shaped second attachment end is configured such that the liquid that the liquid forms a bead on said dish, wherein said dish is configured such that a user can hold an eye drop bottle and attached attachment in a generally vertical position with a bead of liquid from the eye drop bottle positioned on said dish and lower the user's face and eye toward the bead such that contact between the user's eye and the bead of liquid causes the liquid to travel onto the user's eye due to the surface tension of the liquid.

3. The attachment for an eye drop bottle of claim 2, wherein said at least one vent comprises an elongate slot.

4. The attachment for an eye drop bottle of claim 2, wherein said at least one vent comprises a cylindrical vent.

5. The attachment for an eye drop bottle of claim 2, wherein said lumen comprises a plurality of elongate channels of varying cross section extending lengthwise in said lumen and configured to

6. The attachment for an eye drop bottle of claim 2 wherein said lumen is configured in sections, wherein each section is configured with a hydrophobicity or hydrophilicity such that said lumen is configured to facilitate the separation of individual eye drops from said eye drop bottle.

7. The attachment for an eye drop bottle of claim 2 wherein said first attachment end is configured for attachment over the tip of an eyedrop bottle.

8. The attachment for an eye drop bottle of claim 2 wherein said first attachment end is configured for insertion and retention into an eye drop bottle so as to replace a pre-existing tip on the eye drop bottle.

9. An eye drop bottle, said bottle comprising a liquid storage body and an improved dispenser, said improved dispenser comprising:
a first end and a second end separated by an elongate tube section, wherein said elongate tube section defining a lumen configured for the flow of liquid from the eye drop bottle through said first end of said dispense through said lumen to said second end of said dispenser when a user squeezes the eye drop bottle to release an eye drop, wherein said second end is configured in a general dish like shape;
at least one vent positioned in a side of said elongate tube between said first end and said second end and configured to vent said lumen to atmosphere, wherein said vent is configured such that when a user releases depression on said eye drop bottle said vent allows ambient air to enter said lumen and preventing any liquid that has traveled beyond said vent to return via said lumen to said eye drop bottle;
wherein said general dish like shaped second attachment end is configured such that the liquid that the liquid forms a bead on said dish, wherein said dish is configured such that a user can hold an eye drop bottle and attached attachment in a generally vertical position with a bead of liquid from the eye drop bottle positioned on said dish and lower the user's face and eye toward the bead such that contact between the user's eye and the bead of liquid causes the liquid to travel onto the user's eye due to the surface tension of the liquid.

10. The eye drop bottle of claim 9, wherein said dispenser is configured to be detached from said body.

11. The eye drop bottle of claim 9, wherein said at least one vent comprises an elongate slot.

12. The eye drop bottle of claim 9, wherein said at least one vent comprises a cylindrical vent.

13. The eye drop bottle of claim 9, wherein said lumen comprises a plurality of elongate channels of varying cross section extending lengthwise in said lumen and configured to facilitate the separation of individual eye drops from said eye drop bottle.

14. The eye drop bottle of claim 9 wherein said lumen is configured in sections, wherein each section is configured with a hydrophobicity or hydrophilicity such that said lumen is configured to facilitate the separation of individual eye drops from said eye drop bottle.
